(19) United States
(12) Patent Application Publication
(10) Pub. No.: US 2022/0118195 A1
(43) Pub. Date: Apr. 21, 2022

(54) SYSTEM TO FACILITATE A SYRINGE INJECTION

(71) Applicants: Ogheneruno Ekpruke, Katy, TX (US); Stella Ekpruke, Katy, TX (US)

(72) Inventors: Ogheneruno Ekpruke, Katy, TX (US); Stella Ekpruke, Katy, TX (US)

(21) Appl. No.: 17/074,783

(22) Filed: Oct. 20, 2020

(51) Int. Cl. A61M 5/42; A61M 5/52

(52) U.S. Cl. CPC A61M 5/425; A61M 5/178; A61M 2209/084; A61M 5/52

(57) ABSTRACT

A system to facilitate a syringe injection, the system including a skin clamping device, including a first clamp assembly, a second clamp assembly, a joint to connect a center portion of the first clamp assembly to a center portion of the second clamp assembly, such that the first clamp assembly and the second clamp assembly move with respect to each other, and pinch skin of a patient therebetween, a spring disposed between the first clamp assembly and the second clamp assembly to keep the first clamp assembly away from the second clamp assembly, and a hydraulic assembly disposed on at least a portion of the spring and connected to a syringe to compress away from the spring in response to the first clamp assembly moving at least partially toward the second clamp assembly, and decompress toward the spring in response to the first clamp assembly moving at least partially away from the second clamp assembly, and a limb holder to receive a limb of the patient therein.

### BACKGROUND

### 1. Field

The present general inventive concept relates generally to an injection system, and particularly, to a system to facilitate a syringe injection.

### 2. Description of the Related Art

Medical professionals (e.g., doctors, nurses, emergency medical technicians) often have to administer injections as a form of medical treatment. However, the act of providing a shot requires direct contact between the medical professional and skin of a patient. Physical contact is potentially harmful to the medical professional because of a needle stick injury.

The needle stick injury may expose the medical professional to infection such as HIV, Hepatitis B, and/or Hepatitis C, along with other pathogens the patient may be carrying. As such, minimizing physical contact will prevent potential needle stick injuries.

Therefore, there is a need for a system to facilitate a syringe injection that allows the medical professional to hold a portion of skin of the patient.

### SUMMARY

The present general inventive concept provides a system to facilitate a syringe injection.

Additional features and utilities of the present general inventive concept will be set forth in part in the description which follows and, in part, will be obvious from the description, or may be learned by practice of the general inventive concept.

The foregoing and/or other features and utilities of the present general inventive concept may be achieved by providing a system to facilitate a syringe injection, the system including a skin clamping device, including a first clamp assembly, a second clamp assembly, a joint to connect a center portion of the first clamp assembly to a center portion of the second clamp assembly, such that the first clamp assembly and the second clamp assembly move with respect to each other, and pinch skin of a patient therebetween, a spring disposed between the first clamp assembly and the second clamp assembly to keep the first clamp assembly away from the second clamp assembly, and a hydraulic assembly disposed on at least a portion of the spring and connected to a syringe to compress away from the spring in response to the first clamp assembly moving at least partially toward the second clamp assembly, and decompress toward the spring in response to the first clamp assembly moving at least partially away from the second clamp assembly, and a limb holder to receive a limb of the patient therein.

The skin clamping device may further include a syringe holding portion disposed on at least a portion of the first clamp assembly to receive the syringe therein.

The skin clamping device may further include a plurality of jaw covers removably connected to the first clamp assembly and the second clamp assembly to prevent damage to the skin of the patient.

The limb holder may include a first section, including a first body, a clamp connector disposed on at least a portion of an outer surface of the first body to receive at least one of the first clamp assembly and the second clamp assembly therein, and a first arcuate portion arcuately disposed on at least a portion of the first body, such that the first body has an arcuate shape, and a second section, including a second body, and a second arcuate portion arcuately disposed on at least a portion of the second body, such that the second body has an arcuate shape.

The clamp connector may at least partially deform around at least one of the first clamp assembly and the second clamp assembly.

The first section may further include a plurality of protrusion receiving apertures disposed within at least a portion of the first body.

The second section may further include a plurality of protrusions perpendicularly disposed away from a first edge and a second edge of the second body with respect to a direction to be inserted into the plurality of protrusion receiving apertures, such that the first body and the second body collectively form a cylindrical shape.

The second section may further include a cushion disposed on at least a portion of the second arcuate portion to absorb a force thereupon.

### BRIEF DESCRIPTION OF THE DRAWINGS

These and/or other features and utilities of the present generally inventive concept will become apparent and more readily appreciated from the following description of the embodiments, taken in conjunction with the accompanying drawings of which:

FIG. 1 illustrates a front perspective view of a skin clamping device, according to an exemplary embodiment of the present general inventive concept; and

FIG. 2 illustrates an exploded view of a limb holder, according to an exemplary embodiment of the present general inventive concept.

### DETAILED DESCRIPTION

Various example embodiments (a.k.a., exemplary embodiments) will now be described more fully with reference to the accompanying drawings in which some example embodiments are illustrated. In the figures, the thicknesses of lines, layers and/or regions may be exaggerated for clarity.

Accordingly, while example embodiments are capable of various modifications and alternative forms, embodiments thereof are shown by way of example in the figures and will herein be described in detail. It should be understood, however, that there is no intent to limit example embodiments to the particular forms disclosed, but on the contrary, example embodiments are to cover all modifications, equivalents, and alternatives falling within the scope of the disclosure. Like numbers refer to like/similar elements throughout the detailed description.

It is understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

The terminology used herein is for the purpose of describing particular embodiments only and is not intended to be limiting of example embodiments. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements and/or components, but do not preclude the presence or addition of one or more other features, integers, steps, operations, elements, components and/or groups thereof.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art. However, should the present disclosure give a specific meaning to a term deviating from a meaning commonly understood by one of ordinary skill, this meaning is to be taken into account in the specific context this definition is given herein.

### LIST OF COMPONENTS

System 1
Skin Clamping Device 100
First Clamp Assembly 110
First Handle 111
First Aperture 112
First Arm 113
First Jaw 114
Second Clamp Assembly 120
Second Handle 121
Second Aperture 122
Second Arm 123
Second Jaw 124
Joint 130
Spring 140
Hydraulic Assembly 150
Body 151
Clip 152
Syringe Holding Portion 160
Jaw Covers 170
Limb Holder 200
First Section 210
First Body 211
Clamp Connector 212
First Arcuate Portion 213
Protrusion Receiving Apertures 214
Second Section 220
Second Body 221
Second Arcuate Portion 222
Cushion 223
Protrusions 224

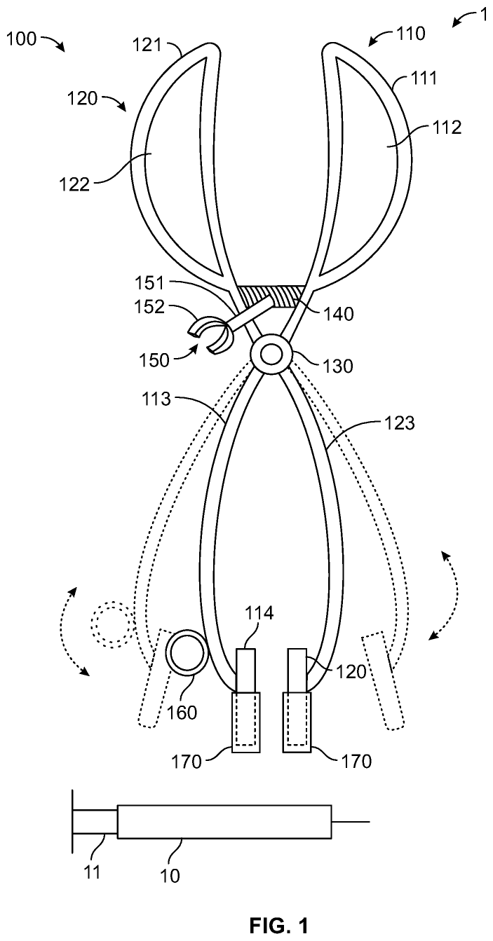

FIG. 1 illustrates a front perspective view of a skin clamping device 100, according to an exemplary embodiment of the present general inventive concept.

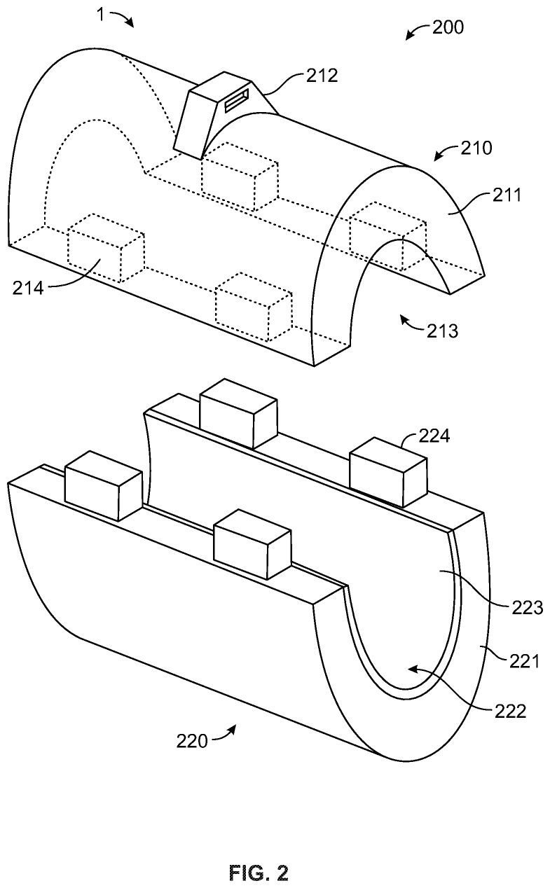

FIG. 2 illustrates an exploded view of a limb holder 200, according to an exemplary embodiment of the present general inventive concept.

A system 1 may be constructed from at least one of metal, plastic, wood, ceramic, and rubber, etc., but is not limited thereto.

The system 1 may include a skin clamping device 100 and a limb holder 200, but is not limited thereto.

The skin clamping device 100 may include a first clamp assembly 110, a second clamp assembly 120, a joint 130, a spring 140, a hydraulic assembly 150, a syringe holding portion 160, and a plurality of jaw covers 170, but is not limited thereto.

Referring to FIG. 1, the skin clamping device 100 is illustrated to be a scissor clamp. However, the skin clamping device 100 may be a trigger-activated bar clamp, a parallel jaw clamp, a pipe clamp, a C-clamp and/or a carriage clamp, a corner clamp, a strap clamp, and a spring clamp, but is not limited thereto.

The first clamp assembly 110 may include a first handle 111, a first aperture 112, a first arm 113, and a first jaw 114, but is not limited thereto.

The first handle 111 may be disposed at a first end on at least a portion of the first arm 113. The first aperture 112 may be disposed on at least a portion of the first handle 111. As such, the first handle 111 and/or the first aperture 112 may facilitate gripping thereof. More specifically, the first aperture 112 may receive at least one finger of a user therethrough.

The first arm 113 may extend away from the first handle 111 with respect to a first direction. Moreover, the first arm 113 may curvedly extend away from the first handle 111, such that the first arm 113 has an S-shape curve with respect to a front view.

Referring again to FIG. 1, the first jaw 114 may be disposed at a second end of the first arm 112. The first jaw 114 may have a partially rounded rectangular shape, such that a first end has a sharp edge and a second end has a rounded edge.

The second clamp assembly 120 may include a second handle 121, a second aperture 122, a second arm 123, and a second jaw 124, but is not limited thereto.

The second handle 121 may be disposed at a first end on at least a portion of the second arm 123. The second aperture 122 may be disposed on at least a portion of the second handle 121. As such, the second handle 121 and/or the second aperture 122 may facilitate gripping thereof. More specifically, the second aperture 122 may receive at least one finger of the user therethrough.

The second arm 123 may extend away from the second handle 121 with respect to the first direction. Moreover, the second arm 123 may curvedly extend away from the second handle 121, such that the second arm 123 has another S-shape curve, such that the second arm 123 may curvedly extend in the S-shape opposite with respect to the S-shape of the first arm 113 with respect to the front view.

Referring again to FIG. 1, the second jaw 124 may be disposed at a second end of the second arm 122. The second jaw 124 may have a partially rounded rectangular shape, such that a first end has a sharp edge and a second end has a rounded edge.

The first arm 113 may be connected to the second arm 123 at a center portion thereof via the joint 130. As such, the first arm 113 and/or the second arm 123 may rotate about the joint 130. Accordingly, the first jaw 114 and/or the second jaw 124 may move in a first lateral direction or a second lateral direction away from each other in response to moving the first handle 111 and/or the second handle 121 in the first lateral direction or the second lateral direction away from each other. Conversely, the first jaw 114 and/or the second jaw 124 may move in the second lateral direction or the first lateral direction toward each other in response to moving the first handle 111 and/or the second handle 121 in the second lateral direction or the first lateral direction toward each other.

Therefore, the first arm 113 may move in a scissor motion with respect to the second arm 123, and vice versa.

Furthermore, the first jaw 114 and/or the second jaw 124 may grip, pinch, hold, and/or grab in response to moving toward each other. Also, the first jaw 114 and/or the second jaw 124 may move (i.e. spread, stretch) at least one object (e.g., skin) apart in response to moving away from each other. As such, the first jaw 114 and/or the second jaw 124 may facilitate access to skin of the user by pinching the skin, such that the skin is elevated to receive a syringe 10 and/or needle for an injection, such as a subcutaneous injection to a patient. Alternatively, the first jaw 114 and/or the second jaw 124 may stretch the skin of the user to receive the syringe 10 and/or needle for the injection, such as an intramuscular injection to the patient.

The spring 140 may be disposed between the first arm 113 and/or the second arm 123 between the first handle 111 and/or the second handle 121, respectively, and the joint 130. Moreover, the spring 140 may keep (i.e. spring bias) the first arm 113 and/or the second arm 123 away from each other. In other words, the first arm 113 and/or the second arm 123 may separate in response to release of the first handle 111 and/or the second handle 121, such that the first arm 113 and/or the second arm 123 return to an original position.

The hydraulic assembly 150 may include a body 151 and a clip 152, but is not limited thereto.

The body 151 may include a hydraulic piston and a pneumatic air cylinder, but is not limited thereto.

The body 151 may be disposed at a first end on at least a portion of the spring 140. The body 151 may compress in response to moving (i.e. squeezing) the first handle 111 and/or the second handle 121 at least partially toward each other, such that the body 151 moves away from the spring 140.

The clip 152 may be disposed on at least a portion of a second end of the body 151. The clip 152 may receive the syringe 10 and/or the needle therein. The clip 152 may move a plunger 11 of the syringe 10 in response to compression of the body 151. Alternatively, the body 151 may decompress (i.e. extend) in response to moving the first handle 111 and/or the second handle 121 at least partially away from each other, such that the body 151 may move the plunger 11 of the syringe 10 out of the syringe 10 in response to decompression of the body 151.

Referring again to FIG. 1, the syringe holding portion 160 is illustrated to be disposed on at least a portion of the first arm 113. However, the syringe holding portion 160 may be disposed on at least a portion of the second arm 123. Also, there may be a plurality of syringe holding portions 160.

The syringe holding portion 160 may receive at least a portion of the syringe 10 and/or the needle therein. More specifically, the syringe holding portion 160 may receive a portion of the syringe 10 therein different from the plunger 11. As such, the syringe holding portion 160 may prevent contact of the syringe 10 with a body of the user.

The plurality of jaw covers 170 may be constructed of silicone and/or plastic.

Each of the plurality of jaw covers 170 may be removably connected to the first jaw 114 and/or the second jaw 124. The plurality of jaw covers 170 may prevent damage to the skin of the patient. Also, the plurality of jaw covers 170 may increase friction against the skin of the patient.

The limb holder 200 may include a first section 210 and a second section 220, but is not limited thereto.

The first section 210 may include a first body 211, a clamp connector 212, a first arcuate portion 213, and a plurality of protrusion receiving apertures 214, but is not limited thereto.

The first body 211 may have a predetermined size (i.e. length, width, and height).

The clamp connector 212 may be disposed on at least a portion of an outer surface of the first body 211. Additionally, the clamp connector 212 may receive at least a portion of the first arm 113 and/or the second arm 123 therein. More specifically, the clamp connector 212 may be flexible (i.e. stretch) to at least partially deform around the first arm 113 and/or the second arm 123 via the first jaw 114 and/or the second jaw 124, respectively.

The first arcuate portion 213 may be arcuately disposed on at least a portion of the first body 211, such that the first body 211 has an arcuate shape.

The plurality of protrusion receiving apertures 214 may be disposed within at least a portion of the first body 211.

The second section 220 may include a second body 221, a second arcuate portion 222, a cushion 223, and a plurality of protrusions 224, but is not limited thereto.

The second body 221 may have a predetermined size (i.e. length, width, and height).

The second arcuate portion 222 may be arcuately disposed on at least a portion of the second body 221, such that the second body 221 has an arcuate shape.

The cushion 223 may include a sponge, a gel, and a silicone substance, but is not limited thereto.

The cushion 223 may be disposed on at least a portion of the second arcuate portion 222. Also, another cushion 223 may be disposed on at least a portion of the first arcuate portion 213. The cushion 223 may absorb a force thereupon and/or prevent injury to a limb (e.g., an arm, a leg) of the patient therein.

The plurality of protrusions 224 may be perpendicularly disposed away from a first edge and/or a second edge of the second body 221 with respect to a direction. Moreover, the plurality of protrusions 224 may correspond to the plurality of protrusion receiving apertures 214. As such, the plurality of protrusions 224 may be removably inserted into the plurality of protrusion receiving apertures 214.

Therefore, collectively, the first body 211 and/or the second body 221 may form a cylindrical shape, such that the limb of the patient may be received therebetween. As such, the first body 211 and/or the second body 221 may prevent movement of the limb of the patient during insertion of the syringe 10 using the skin clamping device 100.

Therefore, the system 1 may facilitate insertion of the syringe 10 using the skin clamping device 100 and/or the limb holder 200 without physical contact by a medical professional.

The present general inventive concept may include a system 1 to facilitate a syringe injection, the system 1 including a skin clamping device 100, including a first clamp assembly 110, a second clamp assembly 120, a joint 130 to connect a center portion of the first clamp assembly 110 to a center portion of the second clamp assembly 120, such that the first clamp assembly 110 and the second clamp assembly 120 move with respect to each other, and pinch skin of a patient therebetween, a spring 140 disposed between the first clamp assembly 110 and the second clamp assembly 120 to keep the first clamp assembly 110 away from the second clamp assembly 120, and a hydraulic assembly 150 disposed on at least a portion of the spring 140 and connected to a syringe 10 to compress away from the spring 140 in response to the first clamp assembly 110 moving at least partially toward the second clamp assembly 120, and decompress toward the spring 140 in response to the first clamp assembly 110 moving at least partially away from the second clamp assembly 120, and a limb holder 200 to receive a limb of the patient therein.

The skin clamping device 100 may further include a syringe holding portion 160 disposed on at least a portion of the first clamp assembly 110 to receive the syringe 10 therein.

The skin clamping device 100 may further include a plurality of jaw covers 170 removably connected to the first clamp assembly 110 and the second clamp assembly 120 to prevent damage to the skin of the patient.

The limb holder 200 may include a first section 210, including a first body 211, a clamp connector 212 disposed on at least a portion of an outer surface of the first body 211 to receive at least one of the first clamp assembly 110 and the second clamp assembly 120 therein, and a first arcuate portion 213 arcuately disposed on at least a portion of the first body 211, such that the first body 211 has an arcuate shape, and a second section 220, including a second body 221, and a second arcuate portion 222 arcuately disposed on at least a portion of the second body 221, such that the second body 221 has an arcuate shape.

The clamp connector 212 may at least partially deform around at least one of the first clamp assembly 110 and the second clamp assembly 120.

The first section 210 may further include a plurality of protrusion receiving apertures 214 disposed within at least a portion of the first body 211.

The second section 220 may further include a plurality of protrusions 224 perpendicularly disposed away from a first edge and a second edge of the second body 221 with respect to a direction to be inserted into the plurality of protrusion receiving apertures 214, such that the first body 211 and the second body 221 collectively form a cylindrical shape.

The second section 220 may further include a cushion 223 disposed on at least a portion of the second arcuate portion 222 to absorb a force thereupon.

Although a few embodiments of the present general inventive concept have been shown and described, it will be appreciated by those skilled in the art that changes may be made in these embodiments without departing from the principles and spirit of the general inventive concept, the scope of which is defined in the appended claims and their equivalents.

1. A system to facilitate a syringe injection, the system comprising:
a skin clamping device, comprising:
a first clamp assembly,
a second clamp assembly,
a joint to connect a center portion of the first clamp assembly to a center portion of the second clamp assembly, such that the first clamp assembly and the second clamp assembly move with respect to each other, and pinch skin of a patient therebetween,
a spring disposed between the first clamp assembly and the second clamp assembly to keep the first clamp assembly away from the second clamp assembly, and
a hydraulic assembly disposed on at least a portion of the spring and connected to a syringe to compress away from the spring in response to the first clamp assembly moving at least partially toward the second clamp assembly, and decompress toward the spring in response to the first clamp assembly moving at least partially away from the second clamp assembly; and
a limb holder to receive a limb of the patient therein.

2. The system of claim 1, wherein the skin clamping device further comprises:
a syringe holding portion disposed on at least a portion of the first clamp assembly to receive the syringe therein.

3. The system of claim 1, wherein the skin clamping device further comprises:
a plurality of jaw covers removably connected to the first clamp assembly and the second clamp assembly to prevent damage to the skin of the patient.

4. The system of claim 1, wherein the limb holder comprises:
a first section, comprising:
a first body,
a clamp connector disposed on at least a portion of an outer surface of the first body to receive at least one of the first clamp assembly and the second clamp assembly therein, and
a first arcuate portion arcuately disposed on at least a portion of the first body, such that the first body has an arcuate shape; and
a second section, comprising:
a second body, and
a second arcuate portion arcuately disposed on at least a portion of the second body, such that the second body has an arcuate shape.

5. The system of claim 4, wherein the clamp connector at least partially deforms around at least one of the first clamp assembly and the second clamp assembly.

6. The system of claim 4, wherein the first section further comprises:
a plurality of protrusion receiving apertures disposed within at least a portion of the first body.

7. The system of claim 6, wherein the second section further comprises:
a plurality of protrusions perpendicularly disposed away from a first edge and a second edge of the second body with respect to a direction to be inserted into the plurality of protrusion receiving apertures, such that the first body and the second body collectively form a cylindrical shape.

8. The system of claim 4, wherein the second section further comprises:
a cushion disposed on at least a portion of the second arcuate portion to absorb a force thereupon.
